(19) United States
(12) Patent Application Publication
(10) Pub. No.: US 2010/0184848 A1
(43) Pub. Date: Jul. 22, 2010

(54) TRANSDERMAL FORMULATIONS OF SYNTHETIC CANNABINOIDS AND NANO COLLOIDAL SILICA

(76) Inventors: William Abraham Wine, (US); Harvey Wine, North York (CA)

Correspondence Address: DAVID S. RESNICK, NIXON PEABODY LLP, 100 SUMMER STREET, BOSTON, MA, 02110-2131, US

(21) Appl. No.: 12/373,632

(22) PCT Filed: Jul. 13, 2007
(86) PCT No.: PCT/CA2007/001272

§ 371 Date: Jan. 4, 2010

Related U.S. Application Data
(60) Provisional application No. 60/830,711, filed on Jul. 14, 2006.

(51) Int. Cl. A61K 31/352; A61K 31/454; A61K 31/436; A61P 25/00

(52) U.S. Cl. 514/454; 514/326; 514/291

(57) ABSTRACT

The present invention relates to pharmaceutical compositions comprising cannabinoids or mimics thereof. In one aspect, the invention provides transdermal formulations comprising cannabinoids or mimics thereof. In another aspect, the invention provides topical formulations comprising cannabinoids or mimics thereof. In one embodiment the formulations and compositions of the invention comprise nano colloidal silica. The formulations of the invention can be used in the therapeutic treatment of many conditions, including wherein the cannabinoids or mimics thereof are known to reduce the excessive neuronal firing characteristic of neuropathic pain. Muscle spasticity is also benefited by these formulations.

### FIELD OF THE INVENTION

This invention relates to transdermal formulations and pharmaceutical compositions of cannabinoids, such as synthetic cannabinoids and mimics thereof and to methods and uses related thereto. In one embodiment, the formulations enable the delivery of the cannabinoid and/or mimic thereof to the site of pain or to sites that play a role in the perception of pain. In another aspect, the invention further relates to transdermal pharmaceutical compositions comprising nanocolloidal silica as the carrier or propellant.

### BACKGROUND OF THE INVENTION

Methods and products for transdermally administering particular chemicals are known in the art. Several U.S. patents have issued for the transdermal application of chemicals, most recently for e.g. nicotine. These patches include reservoirs containing the medicinal compound and rate controlling means, such as membranes of various porosities, which control the flow of the said medicinal compound to and through the skin.

Cannabinoids are a group of terpenophenolic compounds present in Cannabis sativa L. The broader definition of cannabinoids refer to a group of substances that are structurally related to tetrahydrocannabinol (THC) or that bind to cannabinoid receptors. Cannabinoids have been used by man for 10,000 years. They have had many uses including for pain relief. The first cannabinoid licensed for human use was Nabilone, in 1982, a synthetic cannabinoid.

In the case of the administration of therapeutically efficacious cannabinoids, the use of transdermal delivery eliminates the need for an initial pass through the gastrointestinal system and liver, with attendant biochemical breakdown of the therapeutic agent into metabolites. The transdermal application of the therapeutic agent to the site of the pain also concentrates the agent to that site, with secondary transmission through blood and the circulatory system to other receptor locations that play a role in the patient's perception of pain.

With transdermal delivery, some of the active ingredient may reach the brain through the blood and the cerebrospinal fluid, reducing the central neuropathic pain components that may accompany the disease. However, the residual levels of therapeutic agent are not so high as to be toxic or cause side effects such as: paranoia, bizarre ideation, confusion, psychosis, time distortion or motor co-ordination. Also, the potential for “diversion” of the product is extremely low compared to oral formulations, due to their slow, controlled delivery of the therapeutic agent as well as the inherent formulation of the compound, in that they are unlikely to be smoked or swallowed.

Although transdermal cannabinoid formulations have been previously described, such as in Stinchcomb et al., U.S. patent application Ser. No. 11/157034, United States Application Publication No. 20050266061, Published Dec. 1, 2005 and Brooke et al., U.S. Pat. No. 6,328,992, to date, there has not been an optimal transdermal formulation of cannabinoids or mimics thereof.

Brooke et al. U.S. Pat. No. 6,328,992 describes a method for transdermally administering herbal cannabinoids into the bloodstream using a skin patch of a design similar to those used for nicotine, referred to above. However Brooke et al. in the said patent describe certain permeation enhancers and carriers, but those described are not as effective as those comprising the invention herein disclosed. The said Brooke patent U.S. Pat No. 6,328,992, also does not describe a method whereby the delivery method is not affected by the perspiration of the user. The said Brook patent also teaches a method of delivering herbal or herbal extracts from the cannabis plant. The use of these herbal complexes: increases the chance of Central Nervous System (CNS) toxicity with attendant compromise of motor and cognitive functioning complicates or renders impossible the elucidation of the mechanism of action; increases the opportunity for side effects; and prevents the designing of targeted and controlled therapeutic ligand/receptor interactions. The Brooke approach of using herbal and herbal extracts of the cannabis plant prevents the use of these therapies in those jurisdictions where such use is illegal, due mainly to CNS toxicity, referred to above.

What is needed therefore is a method which provides improved delivery formulations of cannabinoids and mimics thereof, including formulations with improved permeation enhancers and carriers; formulations not affected by the perspiration of the user; formulations which permit a lipid soluble medicinal ingredient to be conveyed to dermal, muscle, connective and synovial tissue and principally reside there and secondarily, have the potential to effuse into vascular tissue and thence to the dorsal ganglia and to the brain via the blood and cerebrospinal fluid, respectively.

### SUMMARY OF THE INVENTION

In one embodiment the invention provides a transdermal pharmaceutical composition comprising: therapeutic ligand such as a cannabinoid, such as a synthetic cannabinoid, such as nabilone or a mimic, antagonist, agonist or metabolite or metabolite precurso thereof or pharmaceutical salt thereof and a nanocolloidal silica. In another embodiment it provides a use of nanocolloidal silica as a carrier or propelling agent in a transdermal pharmaceutical composition, such as a cannabinoid transdermal pharmaceutical composition. In another embodiment, the present invention provides a transdermal composition or formulation comprising a cannabinoid, such as a synthetic cannabinoid, such as nabilone or a mimic thereof and DMSO.

In one embodiment, the transdermal pharmaceutical composition of the invention further comprises:

(a) a penetration enhancer (as an optional component);

(b) a solvent; and

(c) a stabilizing agent.

In one embodiment the invention provides a method of transdermally administering synthetic cannabinoids that reduces side effects, allows for the more precise targeting of therapeutic ligand/receptor interactions, and permits more tractable elucidation of the mechanism(s) of action. Because the precise chemical composition of synthetic cannabinoids is known, the chemistry, pharmacology and pharmacokinetics is likewise known; and each active agent can be titrated to the nanogram. Further, in one embodiment the formulation or composition of the invention can be engineered to take maximum advantage of the attributes of that substance's ability to penetrate the dermis to the affected area. Also, the therapeutic agent ligand/receptor interactions can be precisely engineered to reduce CNS toxicity, while maximizing its therapeutic effect.

In one embodiment, the transdermal delivery system, which is the subject of this invention, has a three stage effect. First, since the CB1, CB2, and CBD receptor sites are widely distributed throughout the human body, not only in nervous tissue but also in dermal, connective, muscle and synovial tissue, the largest proportion of the effect is the first effect which is local at the site of application. It is rapid in onset at that site and it is analgesic, antispasmodic, vegetative, anti-infective, anti-inflammatory, and the bulk of it remains at the affected site where it is needed most. Second, later and at lower intensity, the ingredient is conveyed through the vascular system, away from the initial site, to other parts of the body. The principal secondary effect is at the dorsal horn spinal cord ganglia providing analgesia and antispasmodic relief of symptoms. Third, the tertiary action occurs when some of the residual therapeutic agent may pass the blood brain barrier and the ligand or active ingredient attaches to the CB1, CB2 and/or CBD receptor sites. A safe therapeutic benefit is obtained from this binding of a low dose of the residual therapeutic ligand to the central neurotransmitter receptor site(s). The benefit is primarily analgesic, anxiolytic, antispasmodic, opioid sparing, with attendant lack of interference with brain wave activity. However, the residual dose levels are not so high as to cause central nervous system toxicity, adverse central nervous system symptoms or impairment of the ability to operate mechanical equipment, including a motor vehicle.

Each of the three loci of action also has a different response latency, onset, duration and intensity of action, so that the synergistic effect is a cascade of beneficial effects for the patient. In one embodiment, a therapeutic synthetic ligand can be designed to optimize the initial and residual dosages at each of the three loci to optimize the holistic effect of the therapeutic agent. This is not possible where the therapeutic agent is a herbal cannabinoid.

As suggested above, the use of single substances, rather than herbal complexes, also reduces the opportunity of side effects and makes more tractable the elucidation of the mechanisms of action.

The present formulation, herein disclosed, surmounts the difficulties of these prior methods and shows promise, in clinical studies, for the treatment of conditions to include: Fibromyalgia (FM), Carpal Tunnel Syndrome (CTS), Multiple Sclerosis (MS), tendinitis, lower back pain, rotator cuff injury, crush injuries, spinal cord injuries, Post-surgery upper extremity and/or lower extremity crush injuries; Complex Regional Pain Syndrome; TemporoMandibular Joint Disease; Facet Arthritis; Carpal Tunnel Syndrome; Peripheral Diabetic Neuropathy; Brachial Aversion; Cervicogenic Headache; Amputee pain; Phantom Limb Pain; and divers other chronic pain and spasticity conditions.

The invention herein describes a method of delivering synthetic cannabinoids or cannabinoid mimics, herein referred to as a “synthetic” cannabinoid, both lipid soluble and water soluble, through the dermal barrier for the treatment of various medical conditions, including chronic pain. Cannabinoids are a subset of chemical compounds called terpenoids.

Several synthetic cannabinoids have already been formulated, e.g. Nabilone or Dronabinol, and these and other synthetic cannabinoids that are developed may be the active therapeutic agent(s) that are included in the compositions, methods and uses of the present invention.

Additional aspects and advantages of the present invention will be apparent in view of the description which follows. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### DETAILED DESCRIPTION OF THE INVENTION

In one embodiment, the invention provides transdermal formulations comprising nano colloidal silica as a carrier or propellant. In another embodiment, the invention provides transdermal formulations comprising nano colloidal silica and a therapeutic ligand. In another embodiment, the invention provides a topical and/or transdermal pharmaceutical composition comprising a cannabinoid, such as a synthetic cannabinoid, such as nabilone, and nano colloidal silica as a carrier or propellant. In another embodiment, the invention comprises an agonist, antagonist or mimic of a cannabinoid or synthetic cannabinoid and nano colloidal silica as a carrier or propellant. Formulations comprising pharmaceutical acceptable salts of such cannabinoids, mimics, agonists and antagonists, metabolites or metabolite precursors and nano colloidal silica are also provided by the present invention.

In one embodiment the invention provides a transdermal formulation or composition comprising a therapeutic ligand, such as a cannabinoid or synthetic cannabinoid or mimics thereof or agonists or antagonists thereof or pharmaceutically acceptable salts thereof and DMSO. In one embodiment such compositions or formulations further comprise a solvents and stabilizing agents.

In one embodiment, the invention provides formulations and compositions of therapeutic ligands or cannabinoids comprising:

(a) a therapeutic ligand or cannabinoid(e.g. in one embodiment a cannabinoid or synthetic cannabinoid, agonists, antagonists, mimics, metabolites, or metabolite precursors or pharmaceutically acceptable salts thereof or obvious chemical equivalents thereof).

(b) a penetration enhancer (in one embodiment the penetration enhancer, such as DMSO is optional);

(c) a solvent;

(d) a stabilizing agent and

(e) a carrier or propelling agent.

In one embodiment, the therapeutic ligand is present in the composition from about 0.001-1% by weight. In another embodiment, the therapeutic ligand is present in the composition from about 0.01-0.1% by weight. In another embodiment, the therapeutic ligand is present in the composition from about 0.01-0.05% by weight. In another embodiment, the therapeutic ligand is present in the composition from about 0.015-0.030% by weight. In another embodiment it is present in an amount of 0.015-0.025% by weight. In another embodiment it is present in an amount from 0.020-0.025% by weight of the total formulation. In another embodiment it is present in an amount of 14 mg in a 60 gm formulation +/−25% by weight.

In one embodiment, the penetration enhancer is present in an amount of about 0.1% to about 5% or about 1-5% by weight of the composition or formulation. In another embodiment the penetration enhancer is present in an amount of about 2 to about 4% by weight. In another embodiment the penetration enhancer is present in an amount of about 1% to about 3% by weight. In another embodiment it is present in an amount of about 3% by weight of the composition or formulation. In another embodiment it is present in about 2 gm of a 60 gm formulation.

In one embodiment the solvent is present in about 1-5% by weight of the formulation.

In one embodiment, the stabilizing agent is present in an amount of about 80 to about 95% or in another embodiment about 90% by weight of the total composition.

In one embodiment the carrier or propelling agent, such as nano colloidal silica, is present in an amount of about 0.01 to about 10% by weight of the composition or formulation or in another embodiment 0.05 to about 10% by weight. In another embodiment the carrier or propelling agent is present in an amount of about 0.6 to about6% by weight. In another embodiment the carrier or propelling agent is present at about 0.6 to about 5% by weight. In another embodiment the carrier or propelling agent is present at about 1 to about 3% by weight. In another embodiment the carrier or propelling agent is present at about 1 to about 2% by weight +/- 50% by weight of said amount.

“About” as used herein means +/- 10% of the value provided.

In one embodiment a therapeutic ligand is any ligand that can be used for therapeutic purposes.

In one embodiment, the cannabinoid is any synthetic cannabinoid or mimic thereof that can be used for therapeutic purposes. Synthetic cannabinoid as used herein includes a cannabinoid that is structurally related to THC or that binds with a cannabinoid receptor and that is not an herbal cannabinoid or an endogenous cannabinoid. In one embodiment the synthetic cannabinoid is selected from the group consisting of: Nabilone, Sativex, Rimonabant, Dronabinol. CP-55940, HU-210, sr144528, win 55,212-2; Jwh-133 or levonantrodal, or those described in Pertwee, R C. Handb Exp Pharmacol. 2005; (168):1-51 or Stinchcomb et al. (US 20050266061).

The present invention also comprises formulations comprising agonists or antagonists of cannabinoids or mimics thereof.

In one embodiment it is Nabilone or a pharmaceutically acceptable salt, which is not present in the herbal cannabis form. In another embodiment, the synthetic cannabinoid is a pharmaceutically acceptable salt, solvate, metabolite or metabolite precursor thereof.

“Nabilone”, as used herein, is meant to refer to 3-(1,1-dimethylheptyl)-6,6a,7,8,10,10a-hexahydro-1-hydroxy-6,6-dimethyl-9- H-dibenzo[b,d]pyran-9-one as well as to pharmaceutically acceptable salts, solvates, metabolites, and metabolic precursors of 3-(1,1-dimethylheptyl)-6,6a,7,8,10,10a-hexahydro-1-hydroxy-6,6-dimethyl-9H-dibenzo[b,d]pyran-9-one.

“Sativex”, as used herein, is meant to refer to the combination of tetrahydrocannabinol and cannabinol as developed by GW Pharmaceuticals, UK, as well as to pharmaceutically acceptable salts, solvates, metabolites, and metabolic precursors of the components thereof.

“Rimonabant”, as used herein, is meant to refer to 5-(4-Chlorophenyl)-1-(2,4-dichloro-phenyl)-4-methyl-N-(piperidin-1-yl)-1H-pyrazole-3-carboxamide, as well as to pharmaceutically acceptable salts, solvates, metabolites, and metabolic precursors of 5-(4-Chlorophenyl)-1-(2,4-dichloro-phenyl)-4-methyl-N-(piperidin-1-yl)-1H-pyrazole-3-carboxamide.

“Dronabinol”, as used herein, is meant to refer to delta-9-tetrahydrocannabinol, as well as to pharmaceutically acceptable salts, solvates, metabolites, and metabolic precursors of delta-9-tetrahydrocannabinol

“CP-55940”, as used herein, is meant to refer to 2-[(1S,2R,5S)-5-hydroxy-2-(3-hydroxypropyl)cyclohexyl]-5-(2-methyloctan-2-yl)phenol, as well as to pharmaceutically acceptable salts, solvates, metabolites, and metabolic precursors of 2-[(1S,2R,5S)-5-hydroxy-2-(3 -hydroxypropyl)cyclohexyl]-5-(2-methyloctan-2-yl)phenol.

“HU-210”, as used herein, is meant to refer to (6aR-trans-3-(1,1-Dimethylheptyl)-6a,7,10,10a-tetrahydro-1-hydroxy-6,6-dimethyl-6H-dibenzo[b,d]pyran-9-methanol as well as to pharmaceutically acceptable salts, solvates, metabolites, and metabolic precursors of (6aR-trans-3-(1,1-Dimethylheptyl)-6a,7,10,10a-tetrahydro-1-hydroxy-6,6-dimethyl-6H-dibenzo[b,d]pyran-9-methanol.

“sr144528”, as used herein, is meant to refer to the CB2 receptor antagonist described in Portier et al. J. Pharmacol. Exp. Ther. 288: 582-589 (1999) as well as to pharmaceutically acceptable salts, solvates, metabolites, and metabolic precursors of the CB2 receptor antagonist described in the above reference.

“win55,212-2”, as used herein, is meant to refer to (R)-(+)-[2,3-Dihydro-5-methyl-3[(4-morpholinyl)methyl]pyrrolo[1,2,3 -de]-1,4-benzoxazinyl]-(1-naphthalenyl)methanone mesylate salt as well as to pharmaceutically acceptable salts, solvates, metabolites, and metabolic precursors of (R)-(+)-[2,3-Dihydro-5-methyl-3[(4-morpholinyl)methyl]pyrrolo[1,2,3-de]-1,4-benzoxazinyl]-(1-naphthalenyl)methanone mesylate salt.

“Jwh-133”, as used herein, is meant to refer to 3-(1,1-Dimethylbutyl)-1-deoxy-Delta8-tetrahydrocannabinol as well as to pharmaceutically acceptable salts, solvates, metabolites, and metabolic precursors of 3-(1,1-Dimethylbutyl)-1-deoxy-Delta8-tetrahydrocannabinol.

“Levonantrodal” as used herein, is meant to refer to (−)-(6S,6aR,9R,10aR)-5,6,6a,7,8,9,10,10a-octahydro-6-methyl-3-[(R)-1-methyl-4-phenylbutoxy]-1,9-phenanthridinediol 1-acetate, as well as to pharmaceutically acceptable salts, solvates, metabolites, and metabolic precursors of (−)-(6S,6aR,9R, OaR)-5,6,6a,7,8,9,10,10a-octahydro-6-methyl-3-[(R)-1-methyl-4-phenylbutoxy]-1,9-phenanthridinediol 1-acetate.

Mimic of a synthetic cannabinoid as used herein means any synthetic compound that is structurally similar to a synthetic cannabinoid that mimics the effect of a synthetic cannabinoid. In one embodiment, both synthetic cannabinoids and mimics thereof contain a double benzopyran nucleus and are part of the class of chemical compounds called terpenoids. However other aminoalkylindoles have since been shown to exhibit typical cannabinoid pharmacological activity in vitro and in vivo. In one embodiment the cannabinoid mimic is Pravadoline.

In one aspect, agonists and antagonists of said cannabinoid also include, but are not necessarily limited to agonists and antgonists that are structurally similar to the synthetic cannabinoids as noted above.

Suitable cannabinoids, mimics, agonists and antagonists are described in Pertwee, Roger G.; “Cannabinoid Receptor Ligands”, Tocris Bioscience Scientific Review Series No. 16.

In one embodiment “obvious chemical equivalents” include compounds that may have insignificant differences in structure and function to the therapeutic ligand or cannabinoid or synthetic cannabinoid or mimics, antagonists or agonists in question.

Therapeutic purposes as used herein means, any use of the cannabinoid or mimics thereof for therapeutic purposes, such as for the prevention or treatment of neuropathic pain or other uses of cannabinoids, e.g. reducing muscle spasticity in MS patients, as reviewed in Pertwee R G. Pharmacol. Ther. 95:165-174 (2002).

A penetration enhancer as used herein means any compound that augments movement of active compound through the dermis, for instance, that allows a colloidal dispersion of lipid with a non-lipid so it can penetrate body tissues which are composed of lipids and water along with other dermis components. In one embodiment, the penetration enhancer is DMSO, however, any penetration enhancer suitable in and known in the art for transdermal formulations may be used, such a those that allow a colloidal dispersion of a lipid (cannabinoid) with a non lipid so it can penetrate body tissues which are composed of lipids and water along with other dermis components. In one embodiment, DMSO has been shown to be a preferred penetration enhancer and the invention provides a transdermal formulation or composition comprising a therapeutic ligand, e.g. cannabinoid or synthetic cannabinoid, agonists, antagonists, mimics, metabolites, or metabolite precursors or pharmaceutically acceptable salts thereof and DMSO with or without nano colloidal silica.

Solvent as used herein means any substance(s) used to achieve correct texture and adhesion and are well known in the art of transdermal formulations. In one embodiment it is selected from one or more of ethanol, poly ethylene glycol, or any other solvent appropriate for use in transdermal preparations known to those skilled in the art.

Stabilizing agent as used herein refers to compounds or neutral pharmaceutical bases that lowers the rate at which the cannabinoid degrades, under environmental conditions of storage. In one embodiment it minimizes the effect of water or perspiration on the effectiveness of the formulation. In one embodiment it is selected from the group consisting of Lipoderm; Versa; Vitamin E; Cosmetic; HRT; Cliniderm; Dermabase; Glaxal; Vanishing, or any other pharmaceutical base used for topical formulations known to those skilled in the art. In one aspect, the stabilizing agent is not a pharmaceutically active agent.

Carrier or propelling agent as used herein means a compound or formulation that assists in the delivery of the cannabinoid through the dermis. In one embodiment it is nano colloidal silica. Dupont, Inc. manufactures colloidal silica for the purpose of sol-gel polymerization, the production of silica-like surfaces and the synthesis of porous materials. Eka Chemicals, Inc., the largest manufacturer of colloidal silica, produces colloidal silica for use in the electronics, construction and foundry sectors. The use of colloidal silica as a means of bioencapsulation is a recent development. The application of colloidal silica as a bioencapsulation agent or carrier is reviewed in Stephenson C J et al. Int. J. Hyd. Ene. 28:1245-1250 (2003). These applications include the encapsulation of catalysts, antibodies, antigens and live cells. The carrier or propelling agent that is used here is particularly effective in assisting in the transport of cannabinoids across the dermis. The silica nano clusters have extra singlet electron(s) which attach to the cannabinoid molecule, whereby the attachment process then facilitates penetration through the dermis. A suitable nano colloidal silica is manufactured by Royal Body Care Inc., such as under the trade name Microhydrin™ The nano colloidal silica produced by Royal Body Care, Inc. is incorporated into a nutritional supplement. This particular type of nano colloidal silica is produced from silica hydride, an anionic hydride of organo-siliceous compounds. The molecule is comprised of hydrogen anions encased in a silica matrix. Silica hydride is synthesized from silica microclusters. The process is summarized in Stephenson et al. (2003). Microclusters react with hydrogen gas in the presence of two tungsten electrodes to produce the anionic hydride. Silica hydride undergoes further processing in order to produce the nano colloidal form suitable for this invention. However, others would be known to those skilled in the art that have similar properties. Preferred embodiments include similar materials having similar chemical properties or similar sizes.

According to verbal reports of subjects, the formulations prior to the addition of nano colloidal silica were not as highly effective. All of the 42 patients, in our clinical trial, had the nano colloidal silica in the amount specified in the formulation which was dispensed to them. As such, the present invention provides a use of nano colloidal silica as a penetrant, carrier or propelling agent for a transdermal formulation or compositions, such as a therapeutic ligand or cannabinoid formulation. Although nano colloidal silicas have been described before, for instance as an antioxidant and a rehydrator, it has not before been described as a penetration enhancer or carrier or propelling agent for cannabinoids as herein described. Examination of nano colloidal silica's molecular structure suggests a synergy with cannabinoid molecules which permits a superior penetration through the dermis to deeper tissue. In one embodiment, the various radicals on the cannabinoid molecule attach to the nanocolloidal silica, which helps to carry it into the tissue. The electron donor helps to give it a penetration charge tendency. However, on contact with deep tissue this tendency is neutralized. This molecular biochemical synergy is congruent and consistent with patient reports of product efficacy with the formulations and compositions of the present invention.

A person skilled in the art would appreciate upon reading the present description that, the relative portions or amounts of each of the above constituents of the formulations and compositions of the invention will depend upon the actual indications for which the formulation or composition is being administered; e.g. herpetic neuralgia may require less of the penetrant and propellant, than what is required for multiple sclerosis, in order to reach the shallower depth of the affected tissues or the particular cannabinoid receptor or target site. The relative portions or amounts may also depend on the nature of the formulation or composition. In one embodiment the formulation is a transdermal formulation. In one embodiment it is a topical formulation, such as a cream or gel.

In one embodiment, the formulations and compositions of the invention can comprise other pharmaceutically acceptable carriers or excipients known in the art. “Pharmaceutically acceptable carrier” as used herein means a carrier medium which does not interfere with the effectiveness of the biological activity of the active ingredients and which is not toxic to the host or patient. Suitable pharmaceutical carriers or excipients can be found in Remington: The Science and Practice of Pharmacy. 21 ed 2005. (University of Pennsylvania Press). Gennaro A R, et al.

In another embodiment the formulation might comprise a patch.

In one embodiment the formulations and compositions of the invention can be used to deliver a therapeutically effective amount of the therapeutic ligand, e.g. cannabinoid or synthetic cannabinoid, agonists, antagonists, mimics, metabolites, or metabolite precursors or pharmaceutically acceptable salts thereof, to a subject in need thereof. “Therapeutically effective amount” as used herein means as applied to the compositions of the instant invention to the amount of composition sufficient to induce a desired biological result. That result can be alleviation of the signs, symptoms, or causes of a disease, or any other desired alteration of a biological system. It is understood by persons skilled in the art that the effective amount administered to a particular subject may vary and can be dependent on age, weight, sex or other medical or physiological conditions including the condition or severity of the condition to be treated.

In one embodiment, the formulations and compositions of the invention can be applied to the skin and rubbed in. In one embodiment, it is applied twice a day but may be used as required (prn) for breakthrough pain. In one embodiment, the dose per site is about 30 mcg of the formulation of the invention, such as that of Example 1. In one embodiment, larger doses were not necessarily more effective.

In one embodiment the formulations and compositions of the invention can be used in the treatment of neuropathic pain associated with a number of conditions, including, but not necessarily limited to: Fibromyalgia (FM), Carpal Tunnel Syndrome (CTS), Multiple Sclerosis (MS), tendinitis, lower back pain, rotator cuff injury, crush injuries, spinal cord injuries, Post-surgery upper extremities and/or lower extremities crush injuries; Complex Regional Pain Syndrome; TemporoMandibular Joint Disease; Facet Arthritis; Carpal Tunnel Syndrome; Peripheral Diabetic Neuropathy; Brachial Aversion; Cervicogenic Headache; Amputee pain; Phantom Limb Pain; and diverse other chronic pain and spasticity conditions.

The following examples are offered by way of illustration and not by way of limitation.

### Examples

### Example 1

### Transdermal Cannabinoid Formulations

A number of examples of formulations of the invention were made. The formulations were used in the 42 patient open-label study of Example 2 and applied under “Cannacreme-C” 60 gm.

Formulation: Nabilone 14 mg taken from 1 MG Cesamet® capsules, Valeant Pharma, Canada

| | |
|---|---|
| DMSO | 2 cc (2 gm) |
| Ethyl alcohol | 1 cc (1 gm) |
| Propylene glycol | 1 cc (1 gm) |
| Glycerine | 1 cc (1 gm) |
| Nano colloidal Silica | 1 gm |
| in a pharmaceutical base to 60 gm total weight | |

Although the above formulation is specifically described as mentioned above, other formulations can be made in varying component amounts. In one embodiment, the amount of Nabilone can vary +/−25% by weight of the amount stated. In another embodiment the amount of Nano Colloidal Silica can vary +/−50% by weight of the amound indicated. In another embodiment, the other components can vary to achieve desired consistency, or other properties, such as degree of desired drug penetration, flowability, or formulation characteristic variable. In one embodiment the amounts can vary +/−10% by weight.

Formulations with various bases were made and used in the open label study with similar effects. Examples of the bases used are:
Lipoderm
Versa
Vitamin E
Cosmetic HRT
Cliniderm
Dermabase
Glaxal
Vanishing

### Example 2

### Physician-Sponsored, Open Label Equivalent, Compounded Formulation Clinical Study

A 42 patient open label clinical study was performed as a physician sponsored study of patients in treatment for chronic pain with different diagnoses.

Patients ranged in age from 30 to 70 years old and were both male and female.

All patients suffered from chronic neuropathic pain however with varying causal factors or diagnoses including: Multiple Sclerosis; Fibromyalia; Post-surgery upper extremities and/or lower extremities crush injuries; Complex Regional Pain Syndrome; TemporoMandibular Joint Disease; Lower Back Pain; Spinal Cord Injury; Facet Arthritis; Carpal Tunnel Syndrome; Peripheral Diabetic Neuropathy; Brachial Aversion; Spinal Cord Injury; Cervicogenic Headache; Amputee pain; and Phantom Limb Pain. Etc.

The overall trial was 18 months. A ½ tsp (about ¼ gm) dose of the formulation was applied to the site to the site of pain twice a day.

There was good retention in treatment. Only two out of the 42 patients dropped out for reasons unrelated to the treatment.

There were no reports of local site sensitization or irritation from any of the formulations used, utilizing the particular cannabinoid Nabilone.

The results indicate that the formulations were successful in reducing pain. The effect is most pronounced at the site of application. Often these favourable reports have been in patients who have been recalcitrant to previous treatment.

Outcome measures included a patient's own subjective report, clinician's notes, as well as questionnaires such as the McGill Pain Questionnnaire (Melzack R., Pain. 1:277-299, 1975), Fibromyalgia impact Questionnaire (Burckhardt, C. S. et al., J. Rheumatol. 18:728-733, 1991), Visual Analogue Pain Rating Scale (such as reviewed in Wewers M. E. and Lowe N. K. Res. Nursing Health 13:227-236, 1990), and Likert Pain Scales (Likert, R. Arch. Psychol. 140: 55, 1932). Objective electrophysiological monitoring including Quantitative Sensory Testing, Electromyogram, and Nerve Conduction Studies, as required from patient to patient. Over the time that the patients were followed, all patients consistently reported no site sensitization, or irritation, or Adverse Drug Reaction, and consistent reduction in Pains Scores on all of the above indices; Some patients reported as high as a 40% improvement in pain scores over as little as 3 months treatment duration. The highest improvements in the different treatment indications that the drug was used for occurred in post-surgery neuropathic pain, crush injuries and spinal cord injuries. This sharp improvement in this category is counterintuitive to what would generally be expected in this category of patient, which is ordinarily recalcitrant to treatment. This suggests a novel site or mechanism of action.

No major differences were reported between different formulations that employed different penetrating bases.

Prior to the open label study, formulations were made without nano colloidal silica and found not to be as effective as formulations with nano colloidal silica.

All publications and patent applications mentioned in this specification are herein incorporated by reference to the same extent as if each individual publication or patent application was specifically and individually incorporated by reference.

While the foregoing invention has been described in some detail for purposes of clarity and understanding, it will be appreciated by one skilled in the art, from a reading of the disclosure, that various changes in form and detail can be made without departing from the true scope of the invention in the appended claims.

### REFERENCES

Stinchcomb et al., U.S. patent application Ser. No. 11/157034, U.S. Application Publication No. 20050266061, Published Dec. 1, 2005.
Brooke et al., U.S. Pat. No. 6,328,992
Pertwee, R C. Handb Exp Pharmacol. 2005;(168):1-51
Pertwee, Roger G.; “Cannabinoid Receptor Ligands”, Tocris Bioscience Scientific Review Series No. 16
Torcis Reviews No 27 (2006, Torcis Cookson)
Pertwee R G. Pharmacol. Ther. 95:165-174 (2002)
Stephenson C J et al. Int. J. Hyd. Ene. 28:1245-1250 (2003) Remington: The Science and Practice of Pharmacy. 21 ed 2005. (University of Pennsylvania Press). Gennaro A R, et al.
Melzack R., Pain. 1:277-299, 1975
Burckhardt, C. S. et al., J. Rheumatol. 18:728-733, 1991
Wewers M. E. and Lowe N. K. Res. Nursing Health 13:227-236, 1990 Likert, R. Arch. Psychol. 140: 55, 1932

1. A transdermal pharmaceutical composition comprising: a synthetic cannabinoid and nano colloidal silica.

2. The transdermal pharmaceutical composition of claim 1, further comprising:
(a) a penetration enhancer;
(b) a solvent; and
(c) a stabilizing agent.

3. The pharmaceutical composition of claim 1, wherein the synthetic cannabinoid is selected from the group consisting of: Nabilone, Sativex, Rimonabant, Dronabinol, CP- 55940. HU-210, sr144528, win 55,212-2[N], Jwh-133, levonantrodal, and pharmaceutically acceptable salts thereof.

4. The pharmaceutical composition of claim 3 wherein the synthetic cannabinoid is nabilone.
The pharmaceutical composition of claim 1, wherein the synthetic cannabinoid is present in the composition from an amount of about 0.001% to about 1% by weight of the composition.

6. (canceled)

7. (canceled)

8. (canceled)

9. The pharmaceutical composition of claim 1, wherein the nano colloidal silica is present in the composition in an amount of about 0.1% to about 10% by weight of the composition.

10. (canceled)

11. The pharmaceutical composition of claim 2, wherein the penetration enhancer is DMSO.

12. The pharmaceutical composition of claim 2, wherein the penetration enhancer is present in the composition in an amount of about 0.1% to about 5% by weight of the composition.

13. (canceled)

14. The pharmaceutical composition of claim 2, wherein the solvent is selected from the group consisting of: ethanol and propylene glycol.

15. The pharmaceutical composition of claim 2, wherein the solvent is present in the composition in an amount of about 1% to about 5% by weight of the composition.

16. The pharmaceutical composition of claim 2, wherein the stabilizing agent is selected from the group consisting of: Lipoderm;
Versa; Vitamin E; Cosmetic; HRT; Cliniderm; Dermabase; Glaxal; and Vanishing.

17. The pharmaceutical composition of claim 2, wherein the stabilizing agent is present in the composition in an amount of about 80% to about 95% by weight of the composition.

18. The pharmaceutical composition of claim 2, further comprising glycerin.

19. A method of treating neuropathic pain in a subject comprising administering to said subject an effective amount of the pharmaceutical composition of claim 1.

20. The method of claim 14, wherein the neuropathic pain is associated with a condition selected from the group consisting of: Fibromyalgia (FM), Carpal Tunnel Syndrome (CTS), Multiple Sclerosis (MS), tendinitis, lower back pain, rotator cuff injury, crush injuries, spinal cord injuries, Post-surgery upper extremities and/or lower extremities crush injuries; Complex Regional Pain Syndrome; TemporoMandibular Joint Disease; Facet Arthritis; Carpal Tunnel Syndrome; Peripheral Diabetic Neuropathy; Brachial Aversion; Cervicogenic Headache; Amputees; Phantom Limb Pain, chronic pain, and spasticity.

21. (canceled)

22. (canceled)

23. (canceled)

24. A transdermal pharmaceutical composition of claim 1 in the form of a cream, a gel or a patch.
